(19) United States
(12) Patent Application Publication
(10) Pub. No.: US 2021/0038502 A1
(43) Pub. Date: Feb. 11, 2021

(54) S1PR2 ANTAGONISTS FOR TREATING DISEASES INVOLVING ABNORMAL IMMUNE RESPONSES

(71) Applicants: INSERM (Institut National de la Santé et de la Recherche Médicale), Paris (FR); Centre National de la Recherche Scientifique - CNRS, Paris (FR); Université Claude Bernard Lyon 1, Villeurbanne (FR); École Normale Supérieure de Lyon, Lyon cedex 07 (FR)

(72) Inventors: Thierry WALZER, Lyon (FR); Annabelle DROUILLARD, Lyon (FR)

(21) Appl. No.: 16/762,345

(22) PCT Filed: Nov. 7, 2018
(86) PCT No.: PCT/EP2018/080407

§ 371 Date: May 7, 2020

(30) Foreign Application Priority Data
Nov. 8, 2017 (EP) 17306541.8

(51) Int. Cl. A61K 9/00; A61K 9/20

(52) U.S. Cl. CPC A61K 9/0014; A61K 9/007; A61K 9/0053; A61K 45/00; A61K 9/0019; A61K 9/20; A61K 9/0031

(57) ABSTRACT

The present invention pertains to the treatment of diseases inducing or resulting from an abnormal immune response within a body tissue. The present inventors have demonstrated that S1PR2 engagement inhibits chemokine-induced migration of memory T cell subsets. The S1PR2-mediated pathway thus allows for the retention of T cells within a tissue. The inhibition of such a pathway by using selective S1PR2 inhibitors thus represents a promising alternative for the local treatment of several inflammatory diseases. Accordingly, the present invention pertains to a selective antagonist of S1PR2 for use in the treatment of diseases inducing or resulting from an abnormal immune response within a body tissue. The selective antagonist of S1PR2 is administered topically to the tissue in which the abnormal immune response occurs.

### FIELD OF THE INVENTION

The present invention pertains to the use of selective S1PR2 antagonists. More particularly, the present invention relates to selective S1PR2 antagonists for use in the treatment of diseases involving an abnormal activation of immune responses.

### BACKGROUND OF THE INVENTION

Sphingosine-1 phosphate (S1P), is a signaling sphingolipid which binds to five different G-protein coupled receptors (S1P1-5). Extracellular S1P is carried in the body by albumin and other lipoproteins. S1P concentration is maintained at high levels in the blood and lymph and at low levels within tissues (Cyster and Schwab, 2012). S1P1, S1P3 and SIPS are coupled to Gai and provide attractive cues to lymphocytes. A series of studies found that 51P1 allows the egress of T and B cells from SLO to the blood and lymph (Cyster and Schwab, 2012). S1P4 is believed to regulate T cell proliferation and cytokine secretion but not cell migration (Wang et al., 2005). S1PR2 is the only SIP receptor coupled to G12/G13 and signals via Rho instead of Rac like Gai-coupled receptors (Blankenbach et al., 2016). S1PR2 is also known to be insensitive to the effect of FTY720, a supra agonist for all other SIP receptors that induces their internalization (Mandala et al., 2002; Brinkmann et al., 2002).

Few studies have addressed the role of S1P receptors in lymphocytes, and most studies on this topic focused on the role of S1P1.

Recently activated T cells up regulate CD69 expression. CD69 binds surface S1P1 and induces its internalization, trapping activated T cells in inflamed lymph nodes (Shiow et al., 2006) or in non-lymphoid tissues such as the skin (Mackay et al., 2015). Upon T cell receptor stimulation, S1P1 is also down regulated transcriptionally which may contribute to T cells sequestration in lymph nodes (LN). This down regulation is however transient as S1P1 levels increase during differentiation into effector and memory T cells, which is believed to permit egress from secondary lymphoid organs (SLO) by restoring responsiveness to S1P (Matloubian et al., 2004; Pham et al., 2008). S1P responsiveness is also cyclically modulated during lymphocyte recirculation. In particular, lymphocyte S1P1 is down regulated in the blood, up-regulated in lymphoid organs, and down-regulated again in the lymph, in a manner dependent on local S1P concentrations (Lo et al., 2005) and on the GRK2 kinase (Amon et al., 2011).

It was reported that human thymocytes displayed a strong response to S1P in migration assays (Resop et al., 2016) and that tonsil B cell subsets respond to S1P (Sic et al., 2014). Moreover, numerous studies have documented the important lymphopenia induced by the treatment with FTY720 which is now FDA-approved treatment for relapsing multiple sclerosis (Mehling et al., 2008; Vaessen et al., 2006; Pinschewer et al., 2011; Song et al., 2014). FTY720 binds with a higher affinity to S1P1 and although the mechanism of FTY720 action is still debated, it is likely that much of its action comes from its ability to induce S1P1 internalization in lymphocytes. In treated patients, FTY720 induces a quick decrease in peripheral naïve and central memory T cells but it does not affect peripheral TEM cells and NK cells (Mehling et al., 2008; Vaessen et al., 2006; Walzer et al., 2007), which is attributed to a low S1P1 expression on these cell types and a lower sensitivity to FTY720-induced internalization of S1P5 (Jenne et al., 2009). Hence, most of our knowledge of the role of S1P receptors in lymphocyte trafficking results from the study of loss-of-function mutant mouse models or the study of the impact of FTY720 in patients, and the role and regulation of SIP receptors in human leukocytes remains mostly unexplored.

### SUMMARY OF THE INVENTION

The present inventors have demonstrated that S1PR2 engagement inhibits chemokine-induced migration of memory T cell subsets. As S1P levels are high in the lymph, the S1PR2-mediated pathway thus allows for the retention of T cells within a tissue.

Accordingly, the inventors have determined that inhibiting the S1PR2-mediated pathway can help releasing lymphocytes from a tissue in which an inappropriate immune response is observed, i.e. that using S1PR2 selective antagonists allows sending lymphocytes away from a zone where an abnormal activation of the immune response occurs. 51PR2 selective antagonists can thus be used to topically treat inflammatory disorders such as auto-immune disorders, graft rejections, graft versus host disease or allergy.

Nonsteroidal anti-inflammatory (NSAIDs) are the most commonly used anti-inflammatory drugs but have several side effects among which are gastrointestinal ulcers and bleeding (Suleyman et al., 2007).

The identification of novel drugs and therapies for treating inflammatory disorders is thus a constant concern.

The present invention thus pertains to a selective antagonist of S1PR2 for use in the treatment of a disease inducing or resulting from an abnormal immune response within a body tissue.

According to the present invention, the selective antagonist of S1PR2 is administered topically to a tissue in which an abnormal immune response occurs.

Thus, a first aspect of the present invention relates to a selective antagonist of S1PR2 for use in the treatment of a disease inducing or resulting from an abnormal immune response within a body tissue, wherein said antagonist is administered topically to said tissue.

In particular, said disease inducing or resulting from an abnormal immune response within a body tissue is selected from the group consisting of auto-immune diseases, graft rejection, graft versus host disease and allergy.

### DETAILED DESCRIPTION

S1PR2 also referred to as S1P2, EDG-5, H218, AGR16, or lpB2, is a G protein-coupled receptor which binds sphingosine 1-phosphate (SIP).

According to the present invention, a “S1PR2 antagonist” is a natural or synthetic compound capable of stopping the S1PR2-mediated inhibition of the chemokine-induced migration of memory T cell subsets; i.e. a compound blocking the S1PR2 transduction pathway. Such compounds typically act by inhibiting the binding between S1PR2 and S1P or the signaling downstream S1PR2. The Example section of the present invention discloses methods allowing determining whether a compound inhibits the S1PR2-mediated inhibition of chemokine-induced migration of memory T cell subsets. The skilled person thus knows how to identify a “S1PR2-antagonist” in the context of the present invention.

A “selective” S1PR2 antagonist is a compound which specifically inhibits the S1PR2 transduction pathway without inhibiting the other S1P-receptors pathways, i.e. S1PR1, S1PR3, S1PR4 and S1PR5, and particularly the S1PR1 pathway. Typically, the specificity can be measured by testing the effect of those antagonists on surface expression of S1PR expressed in cell lines, or by testing their effect on chemotaxis assays of cell lines expressing individual receptors (see for example the website pubchem.ncbi.nlm.nih.gov/bioassay/616628).

S1PR2 selective antagonists are well known by the skilled person. The prior art comprises multiple examples of S1PR2 selective antagonists. JTE-013 is a well-known S1PR2 selective antagonist (Arikawa et al. 2003; Osada et al. 2002). Kusuni et al (Kusini 2015 and 2016) further described the synthesis of a series of 1,3-bis(aryloxy)benzene derivatives selectively inhibiting S1PR2 activity. The synthesis of S1PR2 antagonists is further disclosed in patent applications published under references WO2013148460, WO2008154470, WO2017148787, EP2762466, EP2980072, KR20140117301 and EP2688593. The content of these patent applications is hereby incorporated by reference into the present specification.

As mentioned above, the present inventors have demonstrated that inhibiting the S1PR2-mediated pathway allows releasing lymphocytes from a tissue in which an inappropriate activation of the immune response is observed. The specific topical administration of a selective S1PR2 antagonist on/into a zone in which an abnormal activation of the immune response occurs would thus be extremely helpful as it would induce the lymphocytes to migrate away from this zone, thereby reducing the local over-activation of the immune response.

In a particular embodiment, said “abnormal activation of the immune response” results from an over-activation of T lymphocytes which results in T cell mediated diseases such as allergic diseases with skin, lung or gut involvement, graft vs host disease inducing colitis, or autoimmune diseases in which T cells induce tissue lesions (for example multiple sclerosis, diabetes etc).

Several pathologies induce or result from an abnormal activation of the immune response, and particularly of T-lymphocytes. In particular, one can cite:
auto-immune diseases which are conditions in which the immune response mistakenly attacks the body. Auto-immune diseases are characterized by an abnormal immune response of the body against substances and tissues normally present in the body;
allergies, said pathologies being engineered by CD4(+) or CD8(+) T lymphocytes, which secrete cytokines in response to activation by allergen-derived peptides;
graft versus host disease, which is a complication following an allogenic transplant wherein the grafted tissue cells attack the host cells a donor resulting in host tissue damage; and
transplant/graft rejection which is a condition following an allogenic transplant wherein the transplanted tissue is rejected by the recipient's immune system, which destroys the transplanted tissue.

A systemic administration of S1PR2 antagonists would likely cause adverse effects. Thus, in the context of the present invention, the S1PR2 selective antagonist is administered topically i.e. applied on or in the specific zone/tissue in which the abnormal immune response occurs. The affected zones are defined as T-cell induced tissue lesions. Typically they are of variable severity ranging from microvascular inflammation to tissue destruction and necrosis.

Thus, another object of the invention is a method for treating a disease inducing or resulting from an abnormal immune response within a body tissue comprising administering to a subject in need thereof a therapeutically effective amount of a S1PR2 selective antagonist, wherein said S1PR2 selective antagonist is administered topically in or on said body tissue.

By a “therapeutically effective amount” is meant a sufficient amount of compound to treat and/or to prevent the disorder.

It will be understood that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; activity of the specific compound employed; the specific composition employed, the age, body weight, general health, sex and diet of the patient; the time of administration, route of administration, and rate of excretion of the specific compound employed; the duration of the treatment; drugs used in combination or coincidental with the specific polypeptide employed; and like factors well known in the medical arts. For example, it is well within the skill of the art to start doses of the compound at levels lower than those required to achieve the desired therapeutic effect and to gradually increase the dosage until the desired effect is achieved.

The antagonists of the present invention, together with one or more conventional adjuvants, carriers, or diluents may be placed into the form of pharmaceutical compositions and unit dosages. The pharmaceutical compositions and unit dosage forms may comprise conventional ingredients in conventional proportions, with or without additional active compounds or principles, and the unit dosage forms may contain any suitable effective amount of the active ingredients commensurate with the intended daily dosage range to be employed. The pharmaceutical compositions may be employed as solids, such as tablets or filled capsules, semisolids, powders, sustained release formulations, or liquids such as solutions, suspensions, emulsions, elixirs, or filled capsules for oral use; or in the form of suppositories for rectal administration; or in the form of sterile injectable solutions for parenteral uses. Formulations containing about one (1) milligram of active ingredient or, more broadly, about 0.01 to about one hundred (100) milligrams, per tablet, are accordingly suitable representative unit dosage forms.

The antagonists of the present invention may be formulated in a wide variety of oral administration dosage forms. The pharmaceutical compositions and dosage forms may comprise compounds of the present invention or pharmaceutically acceptable salts thereof as the active component. The pharmaceutically acceptable carriers may be either solid or liquid. Solid form preparations include powders, tablets, pulls, capsules, cachets, suppositories, and dispersible granules. A solid carrier may be one or more substances which may also act as diluents, flavouring agents, solubilizers, lubricants, suspending agents, binders, preservatives, tablet disintegrating agents, or an encapsulating material. In powders, the carrier generally is a finely divided solid which is a mixture with the finely divided active component. In tablets, the active component generally is mixed with the carrier having the necessary binding capacity in suitable proportions and compacted in the shape and size desired. The powders and tablets preferably contain from about one (1) to about seventy (70) percent of the active compound. Suitable carriers include but are not limited to magnesium carbonate, magnesium stearate, talc, sugar, lactose, pectin, dextrin, starch gelatin, tragacanth, methylcellulose sodium carboxymethylcellulose, a low melting wax, cocoa butter, and the like.

The term “preparation” is intended to include the formulation of the active compound with an encapsulating material as carrier, providing a capsule in which the active component, with or without carriers, is surrounded by a carrier, which is in association with it. Similarly, cachets and lozenges are included. Tablets, powders, capsules, pulls, cachets, and lozenges may be as solid forms suitable for oral administration.

Other forms suitable for oral administration include liquid form preparations including emulsions, syrups, elixirs, aqueous solutions, aqueous suspensions, or solid form preparations which are intended to be converted shortly before use to liquid form preparations. Emulsions may be prepared in solutions, for example, in aqueous propylene glycol solutions or may contain emulsifying agents, for example, such as lecithin, sorbitan monooleate, or acacia. Aqueous solutions can be prepared by dissolving the active component in water and adding suitable colorants, flavours, stabilizers, and thickening agents. Aqueous suspensions can be prepared by dispersing the finely divided active component in water with viscous material, such as natural or synthetic gums, resins, methylcellulose, sodium carboxymethylcellulose, and other well known suspending agents. Solid form preparations include solutions, suspensions, and emulsions, and may contain, in addition to the active component, colorants, flavours, stabilizers, buffers, artificial and natural sweeteners, dispersants, thickeners, solubilising agents, and the like.

The antagonists of the present invention may be formulated for administration as suppositories. Typically, a low melting wax, such as a mixture of fatty-acid glycerides or cocoa butter is first melted and the active component is dispersed homogeneously.

The antagonists of the present invention may be formulated for parenteral administration (e.g., by injection, for example bolus injection or continuous infusion) and may be presented in unit dose form in ampoules, pre-filled syringes small volume infusion or in multi-dose containers with an added preservative. The compositions may take such forms as suspensions, solutions, or emulsions in oily or aqueous vehicles, for example solutions in aqueous polyethylene glycol. Examples of oily or non-aqueous carriers, diluents solvents or vehicles include propylene glycol, polyethylene glycol, vegetable oils (e.g., olive oil, and injectable organic esters (e.g., ethyl oleate), and may contain formulatory agents such as preserving, wetting, emulsifying or suspending, stabilizing and/or dispersing agents. Alternatively, the active ingredient may be in powder form, obtained by aseptic isolation of sterile solid or by lyophilization from solution for constitution before use with a suitable vehicle, e.g., sterile, pyrogen-free water.

The antagonists of the present invention may be formulated for direct administration to the epidermis as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also containing one or more emulsifying agents, stabilizing agents, dispersing agents, suspending agents, thickening agents, or colouring agents. Formulations suitable for topical administration in the mouth include lozenges comprising active agents in a flavoured base, usually sucrose and acacia or tragacanth; pastilles comprising the active ingredient in an inert base such as gelatin and glycerine or sucrose and acacia; and mouthwashes comprising the active ingredient in a suitable liquid carrier. Formulations suitable for topical administration to the eye include eye drops wherein the active ingredient is suspended or dissolved in a suitable carrier, preferably an aqueous solvent.

The antagonists according to the present invention may be formulated for intrapulmonary administration. Suitable formulations for intrapulmonary applications have a particle size typically in the range of 0.1 to 500 microns and are administered by inhalation through the nasal tract or through the mouth in the form of a dry powder or via an aerosol.

According to a particular embodiment, the antagonist according to the present invention is administered, depending on the disease to be treated, as follows:

Allergies and Allergic Diseases

### >Contact Allergies, Eczema and Psoriasis

The tissue to be targeted for treating these pathologies is the epidermis. Indeed, all these pathologies involve the recruitment of inflammatory mediators in the skin.

Thus, the present invention provides a selective S1PR2 antagonist for use in the treatment of disease selected from the group consisting of psoriasis, dermatitis, eczema and contact allergy. In this case, the antagonist can be administered directly to the skin by using one of the formulations cited above. Thus according to this particular embodiment the S1PR2 selective antagonist is administered topically on the skin, on the zone or in near proximity of the zone, where the allergy, eczema and/or psoriasis appears.

### >Asthma, Chronic Obstructive Pulmonary Disease and Chronic Bronchitis

All these pathologies involve an over-activated immune response in the lungs. Thus, in this case, the antagonist according to the invention would be administered by any method allowing the specific targeting of the lungs such as inhaling or spray drying for instance. Thus, according to this particular embodiment, the S1PR2 selective antagonist is administered topically to the lungs.

Transplant Rejection and Graft Versus Host Disease (GVH)

As mentioned above, these pathologies involve improper immune reactions from the host against the transplant or from the transplant against the host.

In this case, the antagonist according to the present invention is administered into the transplant (host against the transplant) or in the injured organs (GVH disease) using appropriate routes (local application, parenteral route, systemic or enteral administration using agents specifically targeting a given tissue).

Auto-Immune Diseases

### >Rheumatoid Arthritis (RA)

In rheumatoid arthritis (RA), the immune system attacks the joints and particularly the synovial tissue. In this case, the antagonist according to the invention would be administered by subcutaneous or intra-articular injection.

Thus, according to this particular embodiment, the S1PR2 selective antagonist is administered subcutaneously or by an intra-articular injection in the inflamed joint.

### >Inflammatory Bowel Diseases

Inflammatory bowel diseases (IBD) describe conditions that cause inflammation in the lining of the intestines. IBD comprise Crohn's disease and ulcerative colitis.

In this case, the antagonist according to the present invention would be formulated for a rectal administration as suppositories or in the form of an oral controlled-release tablet formulated so as to specifically release the antagonist in the intestines.

### >Systemic Lupus Erythematosus (Lupus)

Systemic lupus erythematosus (SLE) is an autoimmune disease in which the body's immune system mistakenly attacks healthy tissue in several parts of the body. SLE can e.g. affect joints, the skin, muscles and/or tissues lining the heart or the lungs. According to this embodiment, the antagonist according to the present invention would be administered topically to the tissue affected by SLE, such as the skin (topically on the skin, on the zone or in near proximity of the zone, where the rash appears), the joints (by an intra-articular injection in the inflamed joint) or the muscles (via an intramuscular injection).

The present invention will be further disclosed in the following examples.

### BRIEF DESCRIPTION OF THE FIGURES

FIG. 1: S1PR2 inhibits CXCL12-induced migration of memory T cell subsets: (A-C) Transwell assays of the migration of the indicated tonsil T cell subsets assessing movement toward CXCL12 (12 ng/ml)+/−S1P (37 nM) as indicated. Results are shown as migration index. Prior to the addition of S1P in the lower chamber, cells were treated or not for 2 h in the top chamber with JTE-013 (1000 nM). Results show the mean+/−SD of 4 independent experiments with N=3 donors. Stars indicate statistical significance when comparing cell migration in the control condition (S1P only) with the conditions with JTE-013 *p<0.05; **p<0.01 (Mann-Whitney tests).

### EXAMPLES

### 1. Material and methods

Patients and Preparation of Human Lymphocyte Suspensions

All material was used after obtaining informed consent and the research was conducted in accordance with the Helsinki Declaration. Human peripheral blood was obtained from healthy donors. Cells were ficollized and then washed several times in complete medium before resuspension in chemotaxis medium. Pediatric tonsils were obtained from patients undergoing tonsillectomy and were cut into pieces using a scalpel and then passed through a 70 μM cell strained to produce a cell suspension. Cells were then ficollized and washed before migration studies.

Chemotaxis Assays

Tonsil lymphocytes or PBMC were suspended in RPMI1640 supplemented with 4 mg/ml fatty acid-free bovine albumin (Sigma, St-Louis, USA). The same medium was used to prepare S1P (Sigma) or CXCL12 (R&D systems, Minneapolis, Minn.) at the indicated concentrations. Cell migration was analyzed in Transwell chambers (Costar, Cambridge, USA) with 5 μm pore-width polycarbonate filters. Pharmacological modulators of S1P receptors FTY720 (Novartis, Basel, Switzerland), JTE-013 (Tocris, Bristol, UK), SEW2871 (Sigma), CYM50358 (Tocris), were used at the indicated concentrations. PBMC or tonsil cells were added to the top chambers of the transwell systems in the presence or absence of the pharmacological inhibitors and incubated at 37° C. for 2 hours. The chemoattractants were then added in the lower chamber and the cells were allowed to migrate for 2 hours. Transmigrated cells were stained for CD3 (UCHT1), CD4 (RPA-T4), CD8 (RPA-T8), CD45RO (UCHL1), CCR7 (G043H7), CD69 (FN50), CD103 (Ber-act8), CD38 (HIT32) and HLA-DR (LN3) and analyzed/counted by flow cytometry (MACSquant, Miltenyi biotec, Bergisch Gladbach, Germany). All antibodies were purchased from BD-Biosciences (San Jose, Calif.), eBioscience (San Diego, Calif.), Beckman-Coulter (Miami, Fla.) or Biolegend (San Diego, Calif.). The migration index was calculated as the ratio between the number of cells migrating in the S1P (or CXCL12) condition and the number of cells migrating in the control (medium only) condition, as measured by flow cytometry. The percentage of input was calculated as the ratio between the number of cells migrating in a given condition and the number of cells used for the chemotaxis assay.

Cell Sorting and RNA Preparation

Tonsil lymphocytes were stained for surface markers (similar stainings as for chemotaxis experiments) and sorted into different subsets using a FACSAria Cell Sorter (Becton-Dickinson, San Jose, USA). Purity of sorted cell populations was over 98% as checked by flow cytometry. Sorted cells were lysed using Trizol reagent (Invitrogen) and RNA was extracted according to the manufacturer's instructions.

Quantitative RT-PCR

We used High capacity RNA-to-cDNA kit (applied biosystem, Carlsbad, USA) to generate cDNA for RT-PCR. PCR was carried out with a SybrGreen-based kit (FastStart Universal SYBR Green Master, Roche, Basel, Switzerland) or SensiFast SYBR No-ROX kit (Bioline) on a StepOne plus instrument (Applied biosystems, Carlsbad, USA) or a LightCycler 480 system (Roche). Primers were designed using the Roche software.

Statistics

Statistical analyses were performed using non parametric two-tailed t-tests run on Excel (Microsoft, USA) or the Prism software (GraphPad). Levels of significance are expressed as p-values (*p<0.05, **p<0.01, ***p<0.001).

### 2. Results and Discussion

Lymphocyte Response to S1P is Regulated During Recirculation Across Lymphoid Organs

We measured the response of freshly isolated human lymphocyte subsets to S1P using transwell migration systems. We first tested the response of PBMC obtained from healthy donors, taking naïve CD4 and CD8 T cells as the prototypic cell types responsive to S1P. T cell subsets were identified by flow cytometry. However, as previously demonstrated in mouse, human blood naïve T cells did not respond to SIP. A prior incubation in culture medium supplemented or not with serum or cytokines did not change this response (data not shown). This unresponsiveness was specific to S1P as blood T cells were highly responsive to chemokines such as CXCL12. Assuming that S1P receptors were desensitized in blood leukocytes as a result of high S1P concentrations in this compartment as described in mouse, we measured the response of freshly isolated tonsil lymphocytes to S1P gradients. Tonsil naïve T cell chemotaxis was strongly increased when S1P gradients were applied, and compared to the control condition without SIP. Maximum migration was obtained with 30-60 nM S1P, in the range of previously published values for mouse T cells. Higher S1P concentrations inhibited this migration, a response typically observed with other G protein-coupled receptors. Both naïve CD4 and CD8 T cells migrated in response to SIP, naïve CD8 T cells displaying the strongest response.

These data show that, like in the mouse (Lo et al., 2005), human lymphocyte response to S1P fluctuates during recirculation, presumably because of receptor internalization in blood lymphocytes.

S1P Inhibit Spontaneous Migration of Memory T Cells

Next, we measured the migration of memory T cell subsets in response to S1P or CXCL12. Blood memory T cells did not respond to S1P but were strongly reactive to CXCL12. Surprisingly, when analyzing the response of tonsil lymphocytes, SiP did not attract TCM and TEM cells towards the lower chamber but rather inhibited their spontaneous migration especially for memory CD4 T cells. TCM cells had an intermediate migratory response between naïve and TEM cells, suggesting that the response to SiP was mediated by two different receptors whose expressions were regulated in opposite ways during T cell differentiation. When expressing the same migration results as “percentage of input”, we noticed that tonsil naïve T cells were rather stationary in the absence of chemotactic signals. Reciprocally, memory T cells of both subsets were constitutively motile, but this migration could be inhibited in a dose-dependent manner by S1P added in the lower chamber. Similar results were obtained with lymph node lymphocytes (data not shown), suggesting that our conclusions on the response of memory T cells to S1P likely apply to those of all SLO. Finally, we also tested the capacity of tonsil T cells to respond to a physiological source of SiP ie fetal calf serum (FCS), diluted at different concentrations. Naïve and memory T cells displayed opposite responses to FCS, naïve T cells being attracted and memory T cell migration being inhibited by S1P.

Altogether, these results show that T cell response to S1P is highly regulated during differentiation and that naïve and TEM cell subsets have opposite responses to SIP. These data also suggest that TEM cells are retained by S1P within SLO while naïve T cells can exit in response to the same signal.

S1PR1 and S1PR2 are Respectively Involved in Naïve and Memory T Cell Response to S1P

To gain insight into the mechanism underlying the different behaviors of naïve and memory T cell subsets in the presence of S1P gradients, we first measured the expression of S1P receptors by semi-quantitative RT-PCR in sorted T cell subsets. S1PR1, S1PR2 and S1PR4 were expressed at high levels in T cells, while S1PR3 was barely detected and S1PR5 was only expressed at low levels in CD8 TEM cells. Of note S1PR1 expression was progressively down regulated upon T cell differentiation into memory cells, while S1PR2 and S1PR4 expression levels remained constitutively expressed. S1PR1 down regulation correlated with KLF2 being strongly down regulated in TEM. KLF2 is know to induce S1PR1 expression in T cells (Carlson et al., 2006).

We then tested the effect of different pharmacological inhibitors of these receptors on the capacity of T cell subsets to respond to SIP. Tonsil naive T cell migration was strongly inhibited by S1PR1 inhibitors FTY720 and SEW2871 but insensitive to the S1PR4 inhibitor CYM50358. The inhibitory effect of SIP on TCM and TEM migration was abrogated by the S1PR2 inhibitors JTE-013. Of note, FTY720 and SEW2871 had a negative impact on memory T cell migration in the presence of S1P while JTE-013 increased naïve T cell migration to S1P, suggesting that S1PR1 and S1PR2 are active in all T cell subsets but that the relative level of each receptor conditions the migratory behavior in response to SIP. Interestingly, when plotting the maximum migration index as a function of S1PR1 expression in the various T cell subsets, we found a very tight linear correlation between both factors, suggesting that S1PR1 expression is the limiting rate factor in S1P-induced migration and that the apparent “repulsion” mediated by S1PR2 only occurs when S1PR1 is expressed at very low levels, ie in memory T cells, especially of the CD4 subset.

Spontaneous Migration of TRM and Activated T Cells is Inhibited by S1P in an S1PR2-Dependent Way

As our results suggested that S1P could be an important tissue-retention signal in human, we next studied the S1P response of resident memory T cells (TRM) and recently activated T cells (act-T cells). In mouse, TRM are believed to be retained in tissues by default of response to SIP. Indeed, TRM express very low levels of KLF2 and S1PR1 (Skon et al., 2013). Moreover, CD69 expression induces S1PR1 internalization, further inhibiting migration towards S1P (Shiow et al., 2006). To test this point in human, we measured the S1P response of human tonsil TRM defined as CD69 positive T cells co-expressing or not CD103. S1P inhibited spontaneous migration of CD4⁺ TRM cells, irrespective of their CD103 expression. Again, the S1PR2 antagonist JTE-013 abrogated this inhibitory effect. For CD8⁺ TRM cells, a similar phenomenon was observed, but only for CD69⁺CD103⁺ cells. CD69⁺CD103⁻CD8⁺ T cells were indeed not really responsive to SIP in this setting.

Upon antigen-mediated activation, T cells are retained within secondary lymphoid organs, presumably to sustain their activation and differentiation through serial interactions with antigen presenting cells. Mechanistically, it was previously reported that mouse T cells lose their reactivity during this phase by down regulating S1PR1 expression (Matloubian et al., 2004). We addressed this point in human, exploiting the fact that tonsils contain act-T cells in a variable proportion, classically defined as CD38⁺HLA-DR⁺. The spontaneous migration of act-CD4⁺ and act-CD8⁺ T cells was strongly inhibited by S1P, an effect that could be again curbed by the S1PR2 antagonist JTE-013. We also measured the expression of S1P receptors in TRM CD69+ and act-T cells. S1PR1 was found to be highly down regulated in act-T cells compared to naïve T cells and undetectable in TRM cells while the level of S1PR2 was similar to that of other T cell subsets.

S1PR2 Engagement Inhibits Chemokine-Induced Migration of Memory T Cell Subsets

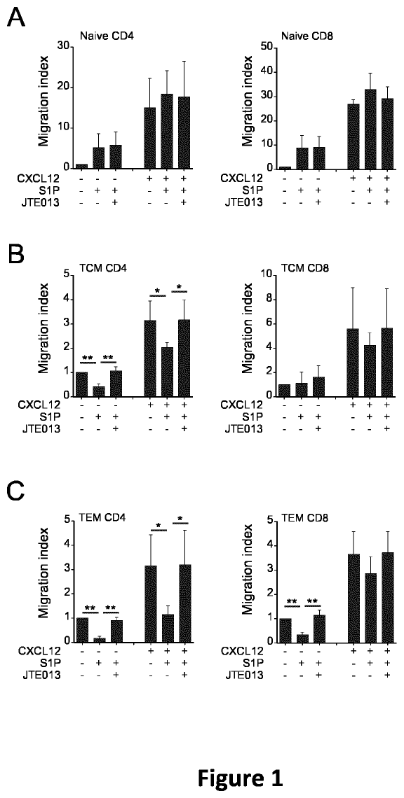

Altogether, our results demonstrate the major role of S1PR2 in human memory T cell response to S1P and suggest a functional antagonism between S1PR1 and S1PR2 in T cell subsets. As lymphocytes are constantly exposed to opposing signals influencing their mobility, we also wanted to test the impact of S1P on the capacity of naïve and memory T cells to respond to chemokine gradients. We therefore measured the migration of tonsil T cells in response to various concentrations of CXCL12, the ligand for CXCR4, a receptor involved in T cell homeostasis (Balabanian et al., 2012) and SLO organization (Hargreaves et al., 2001), in the presence or absence of SiP given at an optimal concentration (37 nM). As shown in FIG. 1A-C, the addition of S1P in the lower chamber slightly increased the migration of naïve CD4 and CD8 T cells but decreased that of TEM cells and to a lesser extent that of TCM cells induced by CXCL12. The latter effect was abrogated by JTE-013 in all conditions, demonstrating that S1PR2 engagement can oppose chemokine-induced migration in memory T cell subsets.

### CONCLUDING REMARKS

Here, we demonstrate that human T cell responsiveness to S1P is regulated at multiple levels. First, as previously demonstrated in mouse (Lo et al., 2005), S1P receptors are desensitized in blood circulating T cells. This is likely the consequence of ligand-induced receptor internalization. All our attempts to restore T cell sensitivity to SiP by pre-incubation in various conditions failed, suggesting that the recycling of S1P receptors on cell surface requires active signals, more complex than the mere deprivation of S1P. Second, the expression of S1P receptors is highly regulated during human T cell differentiation. S1PR1 expression is indeed high in human naïve T cells, likely induced by the transcription factor KLF2 (Carlson et al., 2006) but decreases upon differentiation in TCM and even more in TEM, TRM and act-T cells. By contrast, S1PR2 expression remains stable during differentiation. Hence the ratio between S1PR1 and S1PR2 progressively decreases during T cell differentiation into memory subtypes. This correlated with the migratory behavior of naïve vs memory T cells in the presence of SIP, S1PR1 mediating attraction of naïve T cells while S1PR2 inhibited spontaneous or chemokine-induced memory T cell migration. The affinity of SIP to S1PR2 appears substantially lower than its affinity to S1PR1 (Kd of 27 nM vs Kd of 8 nM for review, see (Kihara, 2014)). This suggests that T cells are attracted towards high S1P concentrations as long as S1PR1 expression is sufficient to overcome S1PR2 activity. Our results suggest that TEM, TRM, act-T cells, and to a lesser extent TCM are retained within SLO through S1PR2 signaling. These data challenge the classical view that S1P is a major exit signal for T lymphocytes. S1P-mediated retention of memory and activated T cells within SLO may favor the re-encounter of antigen presented by dendritic cells. These data may also explain why, as recently shown, TEM cells are very abundant within SLO (Thome et al., 2014). Importantly, memory T cell subsets were found to display higher spontaneous migration than naïve T cells, possibly through constitutive activation of integrins. This intrinsically high mobility may be important for their entry into SLO and to more efficiently scan antigen-presenting cells. It has been postulated that egress structures in SLO like LN may be relatively permissive to T lymphocytes, possibly through egress portals (Pham et al., 2008). In this context, S1PR2 may be important to override the constitutive mobility of memory T cells and promote their retention within SLO. How memory T cell subsets reach the blood circulation remains to be determined. S1P-induced S1PR2 desensitization and attraction by other chemotactic signals such as pro-inflammatory or homeostatic chemokines may allow the egress from SLO. Similar to S1PR1, S1PR2 can be indeed internalized upon stimulation with agonists (Gandy et al., 2013). In Zebrafish, the miles apart mutant, S1PR2 R150H alters the migration of cardiac precursor cells to the midline, a phenomenon due to constitutive desensitization and internalization of S1PR2 (Burczyk et al., 2015). CXCL12 is highly expressed in the medullar region of LN (Hargreaves et al., 2001) and may also contribute to promote entry of memory T cells into lymphatic vessels.

S1PR2 has been previously shown to contribute to accumulation of GC B cells in the central region of the mouse follicle (Green et al., 2011; Wang et al., 2011). Sic et al also showed that human tonsil GC and plasma B cells spontaneous migration was inhibited by S1P (Sic et al., 2014). As these cells express high levels of S1PR2, this was likely to be mediated by S1PR2 although this point required formal testing. Likewise, S1PR2 was shown to be critical for mouse follicular helper T cell retention in germinal centers (Moriyama et al., 2014) and in the proper localization of osteoclast precursors in the bone (Ishii et al., 2010). S1PR2 also inhibits migration in many non-hematopoietic cell types, including vascular endothelial and smooth muscle cells as well as tumor cells (Blankenbach et al., 2016). Our findings therefore corroborate prior reports suggesting counterbalancing roles of S1PR1 and S1PR2 and suggest that the antagonism between S1PR1 and S1PR2 is also important to control the distribution of naïve and memory T cells. Previous studies suggest that S1PR2 usually inhibits S1PR1 signaling by activating Rho and inhibiting Rac (Okamoto et al., 2000; Takashima et al., 2008; Sanchez and Hla, 2004). S1PR1 is also known to signal through Akt (Sanchez and Hla, 2004), an event that has been coupled to SIP responsiveness and actin polymerization in human T cells (Mudd et al., 2013).

### REFERENCES

Throughout this application, various references describe the state of the art to which this invention pertains. The disclosures of these references are hereby incorporated by reference into the present disclosure.
Amon, T. I., Y. Xu, C. Lo, T. Pham, J. An, S. Coughlin, G. W. Dom, and J. G. Cyster. 2011. GRK2-dependent S1PR1 desensitization is required for lymphocytes to overcome their attraction to blood. Science. 333:1898-1903. doi:10.1126/science.1208248.
Balabanian, K., E. Brotin, V. Biajoux, L. Bouchet-Delbos, E. Lainey, 0. Fenneteau, D. Bonnet, L. Fiette, D. Emilie, and F. Bachelerie. 2012. Proper desensitization of CXCR4 is required for lymphocyte development and peripheral compartmentalization in mice. Blood. 119:5722-5730. doi:10.1182/blood-2012-01-403378.
Blankenbach, K. V., S. Schwalm, J. Pfeilschifter, and D. Meyer Zu Heringdorf. 2016. Sphingosine-1-Phosphate Receptor-2 Antagonists: Therapeutic Potential and Potential Risks. Front. Pharmacol. 7:167. doi:10.3389/fphar.2016.00167.
Brinkmann, V., M. D. Davis, C. E. Heise, R. Albert, S. Cottens, R. Hof, C. Bruns, E. Prieschl, T. Baumruker, P. Hiestand, C. A. Foster, M. Zollinger, and K. R. Lynch. 2002. The immune modulator FTY720 targets sphingosine 1-phosphate receptors. J Biol Chem. 277:7.
Burczyk, M., M. D. Burkhalter, T. Blätte, S. Matysik, M. G. Caron, L. S. Barak, and M. Philipp. 2015. Phenotypic regulation of the sphingosine 1-phosphate receptor miles apart by G protein-coupled receptor kinase 2. Biochemistry (Mosc.). 54:765-775. doi:10.1021/bi501061h.
Carlson, C. M., B. T. Endrizzi, J. Wu, X. Ding, M. A. Weinreich, E. R. Walsh, M. A. Wani, J. B. Lingrel, K. A. Hogquist, and S. C. Jameson. 2006. Kruppel-like factor 2 regulates thymocyte and T-cell migration. Nature. 442:299-302. doi:10.1038/nature04882.
Cyster, J. G., and S. R. Schwab. 2012. Sphingosine-1-phosphate and lymphocyte egress from lymphoid organs. Annu. Rev. Immunol. 30:69-94. doi:10.1146/annurev-immunol-020711-075011.
Gandy, K. A. O., M. Adada, D. Canals, B. Carroll, P. Roddy, Y. A. Hannun, and L. M. Obeid. 2013. Epidermal growth factor-induced cellular invasion requires sphingosine-1-phosphate/sphingosine-1-phosphate 2 receptor-mediated ezrin activation. FASEB J. 27:3155-3166. doi:10.1096/fj.13-228460.
Green, J. A., K. Suzuki, B. Cho, L. D. Willison, D. Palmer, C. D. C. Allen, T. H. Schmidt, Y. Xu, R. L. Proia, S. R. Coughlin, and J. G. Cyster. 2011. The sphingosine 1-phosphate receptor S1P₂ maintains the homeostasis of germinal center B cells and promotes niche confinement. Nat. Immunol. 12:672-680. doi:10.1038/ni.2047.
Hargreaves, D. C., P. L. Hyman, T. T. Lu, V. N. Ngo, A. Bidgol, G. Suzuki, Y. R. Zou, D. R. Littman, and J. G. Cyster. 2001. A coordinated change in chemokine responsiveness guides plasma cell movements. J Exp Med. 194:56.
Ishii, M., J. Kikuta, Y. Shimazu, M. Meier-Schellersheim, and R. N. Germain. 2010. Chemorepulsion by blood S1P regulates osteoclast precursor mobilization and bone remodeling in vivo. J. Exp. Med. 207:2793-2798. doi:10.1084/jem.20101474.
Jenne, C. N., A. Enders, R. Rivera, S. R. Watson, A. J. Bankovich, J. P. Pereira, Y. Xu, C. M. Roots, J. N. Beilke, A. Banerjee, S. L. Reiner, S. A. Miller, A. S. Weinmann, C. C. Goodnow, L. L. Lanier, J. G. Cyster, and J. Chun. 2009. T-bet-dependent SIPS expression in NK cells promotes egress from lymph nodes and bone marrow. J Exp Med. 206:81.
Kihara, A. 2014. Sphingosine 1-phosphate is a key metabolite linking sphingolipids to glycerophospholipids. Biochim. Biophys. Acta. 1841:766-772. doi:10.1016/j.bbalip.2013.08.014.
Lo, C. G., Y. Xu, R. L. Proia, and J. G. Cyster. 2005. Cyclical modulation of sphingosine-1-phosphate receptor 1 surface expression during lymphocyte recirculation and relationship to lymphoid organ transit. J Exp Med. 201:301.
Mackay, L. K., A. Braun, B. L. Macleod, N. Collins, C. Tebartz, S. Bedoui, F. R. Carbone, and T. Gebhardt. 2015. Cutting edge: CD69 interference with sphingosine-1-phosphate receptor function regulates peripheral T cell retention. J. Immunol. Baltim. Md 1950. 194:2059-2063. doi:10.4049/jimmunol.1402256.
Mandala, S., R. Hajdu, J. Bergstrom, E. Quackenbush, J. Xie, J. Milligan, R. Thornton, G.-J. Shei, D. Card, C. Keohane, M. Rosenbach, J. Hale, C. L. Lynch, K. Rupprecht, W. Parsons, and H. Rosen. 2002. Alteration of lymphocyte trafficking by sphingosine-1-phosphate receptor agonists. Science. 296:9.
Matloubian, M., C. G. Lo, G. Cinamon, M. J. Lesneski, Y. Xu, V. Brinkmann, M. L. Allende, R. L. Proia, and J. G. Cyster. 2004. Lymphocyte egress from thymus and peripheral lymphoid organs is dependent on SIP receptor 1. Nature. 427:60.
Mehling, M., V. Brinkmann, J. Antel, A. Bar-Or, N. Goebels, C. Vedrine, C. Kristofic, J. Kuhle, R. L. P. Lindberg, and L. Kappos. 2008. FTY720 therapy exerts differential effects on T cell subsets in multiple sclerosis. Neurology. 71:7.
Moriyama, S., N. Takahashi, J. A. Green, S. Hori, M. Kubo, J. G. Cyster, and T. Okada. 2014. Sphingosine-1-phosphate receptor 2 is critical for follicular helper T cell retention in germinal centers. J. Exp. Med. 211:1297-1305. doi:10.1084/jem.20131666.
Mudd, J. C., P. Murphy, M. Manion, R. Debernardo, J. Hardacre, J. Ammori, G. A. Hardy, C. V. Harding, G. H. Mahabaleshwar, M. K. Jain, J. M. Jacobson, A. D. Brooks, S. Lewis, T. W. Schacker, J. Anderson, E. K. Haddad, R. A. Cubas, B. Rodriguez, S. F. Sieg, and M. M. Lederman. 2013. Impaired T-cell responses to sphingosine-1-phosphate in HIV-1 infected lymph nodes. Blood. 121:2914-2922. doi:10.1182/blood-2012-07-445783.
Mueller, S. N., T. Gebhardt, F. R. Carbone, and W. R. Heath. 2013. Memory T cell subsets, migration patterns, and tissue residence. Annu. Rev. Immunol. 31:137-161. doi:10.1146/annurev-immunol-032712-095954.
Okamoto, H., N. Takuwa, T. Yokomizo, N. Sugimoto, S. Sakurada, H. Shigematsu, and Y. Takuwa. 2000. Inhibitory regulation of Rac activation, membrane ruffling, and cell migration by the G protein-coupled sphingosine-1-phosphate receptor EDGS but not EDG1 or EDG3. Mol. Cell. Biol. 20:9247-9261.
Pham, T. H. M., T. Okada, M. Matloubian, C. G. Lo, and J. G. Cyster. 2008. S1P1 receptor signaling overrides retention mediated by G alpha i-coupled receptors to promote T cell egress. Immunity. 28:33.
Pinschewer, D. D., V. Brinkmann, and D. Merkler. 2011. Impact of sphingosine 1-phosphate modulation on immune outcomes. Neurology. 76:S15-19. doi:10.1212/WNL.0b013e31820d9596.
Resop, R. S., M. Douaisi, J. Craft, L. C. M. Jachimowski, B. Blom, and C. H. Uittenbogaart. 2016. Sphingosine-1-phosphate/sphingosine-1-phosphate receptor 1 signaling is required for migration of naive human T cells from the thymus to the periphery. J. Allergy Clin. Immunol. 138:551-557. e8. doi:10.1016/j.jaci.2015.12.1339.
Sallusto, F., D. Lenig, R. Førster, M. Lipp, and A. Lanzavecchia. 1999. Two subsets of memory T lymphocytes with distinct homing potentials and effector functions. Nature. 401:708-712. doi:10.1038/44385.
Sanchez, T., and T. Hla. 2004. Structural and functional characteristics of SIP receptors. J Cell Biochem. 92:913-922. doi:10.1002/(ISSN)1097-4644.
Shiow, L. R., D. B. Rosen, N. Brdicková, Y. Xu, J. An, L. L. Lanier, J. G. Cyster, and M. Matloubian. 2006. CD69 acts downstream of interferon-alpha/beta to inhibit S1P1 and lymphocyte egress from lymphoid organs. Nature. 440:4.
Sic, H., H. Kraus, J. Madl, K.-A. Flittner, A. L. von Miinchow, K. Pieper, M. Rizzi, A.-K. Kienzler, K. Ayata, S. Rauer, B. Kleuser, U. Salzer, M. Burger, K. Zirlik, V. Lougaris, A. Plebani, W. Römer, C. Loeffler, S. Scaramuzza, A. Villa, E. Noguchi, B. Grimbacher, and H. Eibel. 2014. Sphingosine-1-phosphate receptors control B-cell migration through signaling components associated with primary immunodeficiencies, chronic lymphocytic leukemia, and multiple sclerosis. J. Allergy Clin. Immunol. 134:420-428. doi:10.1016/j.jaci.2014.01.037.
Skon, C. N., J.-Y. Lee, K. G. Anderson, D. Masopust, K. A. Hogquist, and S. C. Jameson. 2013. Transcriptional downregulation of Slprl is required for the establishment of resident memory CD8+ T cells. Nat. Immunol. 14:1285-1293. doi:10.1038/ni.2745.
Song, Z.-Y., R. Yamasaki, Y. Kawano, S. Sato, K. Masaki, S. Yoshimura, D. Matsuse, H. Murai, T. Matsushita, and J. Kira. 2014. Peripheral blood T cell dynamics predict relapse in multiple sclerosis patients on fingolimod. PloS One. 10:e0124923. doi:10.1371/journal.pone.0124923.
Steinert, E. M., J. M. Schenkel, K. A. Fraser, L. K. Beura, L. S. Manlove, B. Z. Igyártó, P. J. Southern, and D. Masopust. 2015. Quantifying Memory CD8 T Cells Reveals Regionalization of Immunosurveillance. Cell. 161:737-749. doi: 10.1016/j.cell.2015.03.031.
Takashima, S.-I., N. Sugimoto, N. Takuwa, Y. Okamoto, K. Yoshioka, M. Takamura, S. Takata, S. Kaneko, and Y. Takuwa. 2008. G12/13 and Gq mediate S1P2-induced inhibition of Rac and migration in vascular smooth muscle in a manner dependent on Rho but not Rho kinase. Cardiovasc. Res. 79:689-697. doi:10.1093/cvr/cvn118.
Thome, J. J. C., N. Yudanin, Y. Ohmura, M. Kubota, B. Grinshpun, T. Sathaliyawala, T. Kato, H. Lerner, Y. Shen, and D. L. Farber. 2014. Spatial map of human T cell compartmentalization and maintenance over decades of life. Cell. 159:814-828. doi:10.1016/j.cell.2014.10.026.
Vaessen, L. M. B., N. M. van Besouw, W. M. Mol, J. N. M. Ijzermans, and W. Weimar. 2006. FTY720 treatment of kidney transplant patients: a differential effect on B cells, naïve T cells, memory T cells and NK cells. Transpl. Immunol. 15:281-288. doi:10.1016/j.trim.2006.02.002.
Walzer, T., L. Chiossone, J. Chaix, A. Calver, C. Carozzo, L. Garrigue-Antar, Y. Jacques, M. Baratin, E. Tomasello, and E. Vivier. 2007. Natural killer cell trafficking in vivo requires a dedicated sphingosine 1-phosphate receptor. Nat Immunol. 8:44.
Wang, W., M. H. Graeler, and E. J. Goetzl. 2005. Type 4 sphingosine 1-phosphate G protein-coupled receptor (S1P4) transduces S1P effects on T cell proliferation and cytokine secretion without signaling migration. FASEB J. Off. Publ. Fed. Am. Soc. Exp. Biol. 19:1731-1733. doi: 10.1096/fj0.05-3730fje.
H. Silleyman, Berna Demircan, Yalcin Karagoz, 2007. Anti-inflammatory and side effects of cyclooxygenase inhibitors. Pharmacological Reports, 2007, 59, 247-258
Arikawa K, Takuwa N, Yamaguchi H, Sugimoto N, Kitayama J, Nagawa H, Takehara K, Takuwa Y. Ligand-dependent inhibition of B16 melanoma cell migration and invasion via endogenous S1P2 G protein-coupled receptor. Requirement of inhibition of cellular RAC activity. J Biol Chem 278: 32841-32851, 2003.
Osada M, Yatomi Y, Ohmori T, Ikeda H, Ozaki Y. Enhancement of sphingosine 1-phosphate-induced migration of vascular endothelial cells and smooth muscle cells by an EDG-5 antagonist. Biochem Biophys Res Commun 299: 483-487, 2002.
Kensuke Kusumi, Koji Shinozaki, Toshiya Kanaji, Haruto Kurata, Atsushi Naganawa, Kazuhiro Otsuki, Takeshi Matsushita, Tetsuya Sekiguchi, Akito Kakuuchi, Takuya Seko, Bioorganic & Medicinal Chemistry Letters 25 (2015) 1479-1482.
Kensuke Kusumi, Koji Shinozaki, Toshiya Kanaji, Haruto Kurata, Atsushi Naganawa, Kazuhiro Otsuki, Takeshi Matsushita, Tetsuya Sekiguchi, Akito Kakuuchi, Takuya Seko, Bioorganic & Medicinal Chemistry Letters 25 (2015) 4387-4392.
Kensuke Kusumi, Koji Shinozaki, Toshiya Kanaji, Haruto Kurata, Atsushi Naganawa, Kazuhiro Otsuki, Takeshi Matsushita, Tetsuya Sekiguchi, Akito Kakuuchi, Takuya Seko, Bioorganic & Medicinal Chemistry Letters 26 (2016) 1209-1213.

1. A method for the treatment of a disease inducing or resulting from an abnormal immune response within a body tissue, said method comprising administering topically to said body tissue a therapeutically effective amount of a selective antagonist of sphingosine-1-phosphate receptor 2 (S1PR2).

2. The method according to claim 1, wherein said disease inducing or resulting from an abnormal immune response within a body tissue is selected from the group consisting of auto-immune diseases, graft rejection, graft versus host disease and allergies.

3. The method according to claim 1, wherein said disease inducing or resulting from an abnormal immune response within a body tissue is selected from the group consisting of contact allergies, eczema and psoriasis, and wherein said antagonist is administered topically to skin ata zone or in near proximity of the zone where the contact allergies, eczema and/or psoriasis appears.

4. The method according to claim 1, wherein said disease inducing or resulting from an abnormal immune response within a body tissue is selected from the group consisting of asthma, chronic obstructive pulmonary disease and chronic bronchitis, and wherein said antagonist is administered topically to the lungs.

5. The method according to claim 1, wherein said disease inducing or resulting from an abnormal immune response within a body tissue is a transplant rejection, and wherein said antagonist is administered topically into the transplant.

6. The method according to claim 1, wherein said disease inducing or resulting from an abnormal immune response within a body tissue is a graft versus host disease, and wherein said antagonist is administered topically to organs injured by grafted tissue cells.

7. The method according to claim 1, wherein said disease inducing or resulting from an abnormal immune response within a body tissue is rheumatoid arthritis, and wherein said antagonist is administered subcutaneously or by an intra-articular injection of an inflamed joint.

8. The method according to claim 1, wherein said disease inducing or resulting from an abnormal immune response within a body tissue is an inflammatory bowel disease, and wherein said antagonist is administered rectally or in the form of an oral controlled-release tablet targeting the intestines.

9. The method according to claim 1, wherein said disease inducing or resulting from an abnormal immune response within a body tissue is systemic lupus erythematosus, and wherein said antagonist is administered topically to tissue affected by the systemic lupus erythematosus.
